(19) United States
(12) Patent Application Publication
(10) Pub. No.: US 2002/0172709 A1
(43) Pub. Date: Nov. 21, 2002

(54) Medical dressing comprising an antimicrobial silver compound and a method for enhancing wound healing

(76) Inventors: Brian Nielsen, Goerloese (DK); Trine Wulff, Humlebaek (DK)

Correspondence Address: JACOBSON HOLMAN, PROFESSIONAL LIMITED LIABILITY COMPANY, 400 SEVENTH STREET, N.W., WASHINGTON, DC, 20004, US

(21) Appl. No.: 09/923,913

(22) Filed: Aug. 8, 2001

(30) Foreign Application Priority Data
Mar. 30, 2001 (DK) PA 2001 00535

(51) Int. Cl. A61L 15/00

(52) U.S. Cl. 424/445

(57) ABSTRACT

A medical dressing comprising an antimicrobial silver compound and a method for enhancing wound healing.

A medical dressing comprising a silver compound and being capable of releasing antimicrobial silver ion activity to a wound and, at the same time, being capable of absorbing wound exudate and also degrading enzymes from the wound initiates healing of chronic ulcers which for a long period has not responded by healing as a result of treatment with known wound dressings.

### BACKGROUND OF THE INVENTION

1. Field of the Invention

The present invention relates to a medical dressing comprising a complex of silver and being capable of releasing antimicrobial silver ion activity to a wound, a method for preparing such dressing, and a method for treating a human being.

The primary therapy of chronic wounds is to treat the underlying conditions causing the wound, such as venous disease etc. However, other treatment targets also seem relevant when trying actively to promote healing of recalcitrant ulcers.

Burns, leg ulcers, diabetic foot ulcers and pressure sores are all often more or less colonised or infected. The load of bacteria causes a risk of severe infection which may lead to amputation of parts of or whole extremities and eventually death e.g. due to sepsis. To avoid this, systemic antibiotic treatment is widely used in connection with the treatment of such wounds, which as a side effect create resistant bacteria species. Therefore, several antibacterial wound dressings have been developed for replacing or assisting therapy with systemic antibiotics. Some of these products claim that antimicrobial agents are delivered to the wound to avoid or treat infection.

2. Description of the Related Art

The antiseptic activity of silver compounds is a well known property which has been utilized for many years. The bacteriostatic and fungistatic effect is caused by the silver ion and a simple compound which has been used clinically is for instance silver nitrate.

Bacteriostatics based on the silver ion are further used in various medical devices. One example of such application is the use in the wound dressing sold by Johnson & Johnson under the trademark Actisorb® which is an activated charcoal cloth dressing. Another example is the wound dressing sold under the trademark EZ-Derm by Genetic Laboratories which dressing is a modified pigskin impregnated with a soluble silver compound intended for treatment of burns. A number of patents disclose compositions or devices showing antiseptic properties based on contents of silver compounds. EP 272 149 B1 discloses a medical dressing of the ‘hydrocolloid’ type containing and releasing active components. Silver chloride is a specific antiseptically acting compound mentioned in this patent.

However, there is still a problem in the handling of chronic ulcers often do not respond by healing when treated with known wound dressings comprising antibacterial agents. Research has shown, that excess of proteolytic enzymes is found in wound tissue in chronic ulcers compared to acute wounds.

Thus, in practice it does not seem effective only to deliver an anti-microbial agent in such an amount, that the risk of infection is minimised.

Thus there still seems to be a need for a moist wound healing product comprising an anti-microbial agent in such an amount, that not only the risk of infection is minimised but also the wound healing of such wounds are actively being promoted.

Absorbing wound dressings are well known for use in connection with absorption of exudate from exuding wounds in order to reduce the amount of liquid.

However, it is also well-known that a moist wound healing environment should be retained to support the wound healing process as compared to traditional treatment under dry conditions. Moist conditions are favorable i.a. to avoid energy used for scab formation etc.

Now it has surprisingly been found that the healing of chronic ulcers may be initiated, even after long-lasting lack of response to treatment with known dressings for wound treatment.

### SUMMARY OF THE INVENTION

The present invention relates to a medical dressing comprising a silver compound and being capable of absorbing wound exudate.

Furthermore, the invention relates to a method of enhancing healing of a wound comprising applying to the wound a dressing being capable of delivering an anti-microbially effective amount of silver ion activity to the wound bed and also being capable of removing wound exudate.

### DETAILED DESCRIPTION OF THE PRESENT INVENTION

The present invention relates to a medical dressing comprising a silver compound and being capable of releasing antimicrobial silver ion activity to a wound and, at the same time, being capable of absorbing wound exudate and also degrading enzymes from the wound.

Such a dressing has surprisingly been found to initiate healing of chronic ulcers which for a long period has not responded by healing as a result of treatment with known wound dressings.

A dressing of the invention typically comprises a substantially water-impervious layer or film and a skin-friendly adhesive matrix and, in the form of a separate constituent or in the form of hydrocolloid particles distributed in the adhesive matrix, an absorbing moiety and a silver compound.

The present invention relates to a wound care product for use in moist wound healing. Further, the wound care product transports exudate away from the wound bed by absorption into the wound dressing. Still further, the wound care product releases anti-microbial activity to the wound bed in such an amount that the risk of infection in the wound bed is minimised. Altogether, a wound dressing of the invention has been found to accelerate the wound healing process as compared to a standard moist wound care healing product.

It has surprisingly been observed, that wound dressings combining moist wound healing, absorption of wound exudate and continuous high release of silver ions has a remarkable cleaning and healing promoting effect on wounds with delayed healing, also compared to the effect when using similar wound dressings without release of silver.

It has been found that excess of matrix metallo-proteinases is found in chronic ulcers compared to acute wounds.

It is assumed that healing in many wounds is delayed due to excess of matrix metallo-proteinases excreted from bacteria. Some bacteria species, such as Pseudomonas aeruginosa, release significant amounts of matrix metallo-proteinases, resulting in tissue destruction.

Without limiting the invention to any specific hypothesis it is believed that the dressing according to the invention causes a wound healing effect through reduction of the activity of degrading enzymes, partially by inhibiting the activity of bacteria and thus the secretion of matrix metallo-proteinases etc. and partially by removing these enzymes together with wound exudate by absorption.

Thus, it does not seem effective only to deliver an anti-microbial agent in such an amount, that the risk of infection is minimised, but a further measure has to be taken in order that the wound bed is cleaned.

It is believed that one significant reason that healing in many wounds is delayed is excess of proteolytic enzymes such as matrix metallo-proteinases secreted from bacteria as well as the enzymes arriving from the ulcer itself (matrix metallo-proteinases and enzymes from the inflammation burst, e.g. elastase). Some bacteria species, such as Pseudomonas aeruginosa, release significant amounts of matrix metalloproteinases, resulting in tissue destruction.

It is believed that a balanced removal of exudate is important in wounds with delayed healing, as excess of matrix metalloproteinases and other destructive substances from the wound bed could thus continuously be transported away from the wound bed.

Recently developed active therapies for chronic wounds deliver growth factors or matrix metalloproteinase inhibitors to the wound bed. Some challenges for these kinds of products are that biochemical feedback mechanisms will up- or down regulate the intrinsic delivery of these substances as they are supplied locally, and furthermore, a cocktail of biochemical factors is probably needed for such treatment approach.

It is thought that one reason that combining removal of exudate using an absorbing dressing, a moist wound healing environment and a continuous high release of silver ions promote healing is that a sort of threshold value is surpassed triggering the wound healing.

Thus, removal of exudate actively decreases the amount of proteolytic enzymes in the wound bed and release of silver reduces the amount of bacteria, which leads to decreased formation of matrix metallo-proteinases etc. from this source.

All three features support wound healing, but when treating wounds with delayed healing it seems necessary to balance the three features to pass a threshold and enable the wound healing to proceed, as treatment with either moist wound healing, exudate handling or antibacterial therapy alone in many cases not is sufficient to achieve a biochemically acceptable environment to kick start the healing process in a wound with delayed healing.

A medical dressing according to the invention preferably comprises the silver compound in the form of a complex stabilizing the silver against reduction to free silver. Such stabilization ensures that the activity of silver is not lost during storage and furthermore reduces the risk of immediate inactivation of the silver ions on contact with the wound fluid.

Suitable complexes of silver for use in the dressings of the invention are complexes comprising silver and an element of Group IVa of the periodic system. The complex used in accordance with the present invention may preferably comprise titanium, zirconium of hafnium, and it is especially preferred that the silver is in the form of complex with zirconium.

The complex is suitably a phosphate complex not having adverse effect when in contact with open wounds. Such complex preferably also comprises a further cation such as an alkali metal ion e.g. lithium, sodium, or potassium, preferably sodium.

A silver sodium hydrogen zirconium phosphate complex has proven to be especially suitable for the purpose of the present invention.

Other suitable complexes of silver for use in the dressings of the invention are silver in the form of a complex with a primary, secondary or tertiary amine or amino alcohol.

The amine being used in the compositions of the invention are suitably a primary, secondary or tertiary lower alkyl amine or amino alcohol having a free lone pair of electrons.

A lower alkyl amine is preferably selected from mono, di or tri methyl, ethyl, propyl or butyl amines or mixtures thereof.

A lower alkyl amino alcohol is preferably selected from mono, di or tri methyl ethyl or propyl aminoalcohols or mixtures thereof.

A suitable silver complex is a complex with 5,5-dimethyl hydantoin.

The load of silver is preferably sufficiently high to ensure a steady and high release of silver during the effective time of use of the dressing.

Preferred release of silver is above 200 micrograms per cm², and may be above 300 or even above 400 micrograms per cm² of dressing when determined as disclosed below.

Lower release of silver may show the desired effect provided that the absorbing capacity is sufficiently high, e.g. higher than 0.09 grams per cm² dressing. Thus, the release may e.g. be in the range of 50-10000 micrograms per cm² dressing, more preferred in the range of 100-4000 micrograms per cm² dressing and most preferred in the range of 200-2000 micrograms per cm² dressing. Such silver release ensures a sufficient concentration of silver in the wound to give rise to a dressing kick-starting the beginning of healing of chronic wounds.

The dressings of the invention preferably comprise an absorbing moiety in the form of an individual part of the dressing or in the form of a discontinuous phase distributed in an adhesive matrix.

Thus, the absorbing constituents may be in the form of hydrocolloid particles distributed in an adhesive matrix. Alternatively, the absorbing constituents are in the form of an element of an absorbing foam material.

It is very suitable if the absorbing constituent is in the form of an element of an alginate material.

An absorbing foam material is preferably a polyurethane foam material which may fairly simply be tailored to the purpose of the present invention with respect to release of silver and absorption of exudate.

An alginate material may e.g. be a suitable commercially available material showing a sufficient absorption capacity and being capable of containing and releasing silver in the desired amounts. Such a material is e.g. the material disclosed in WO 95/05204.

A dressing of the invention comprising an alginate moiety may suitably be without a substantially water-impervious layer or film and be used in accordance with the conventional use of corresponding alginate dressings without silver. hydrogel of the invention will typically not comprise a substantially water-impervious layer or film but is used in same manner as a conventional gel.

An absorption capacity of more than 0.09 gram per cm² dressing, more preferred more than 0.12 grams per cm² dressing and most preferred more than 0.15 grams per cm² dressing is believed to gives rise to a balanced removal of exudate and accompanying proteases enhancing the healing of chronic wounds.

In a preferred embodiment of the invention, the silver is essentially homogeneously distributed in the adhesive matrix and/or the absorbing moiety.

A dressing of the invention comprising a separate absorbing element is suitably located in the form of an “island” encircled by an adhesive border. The dressing may have any appropriate shape such as circular, oval, square or rectangular.

A preferred embodiment of the invention is in the form of a dressing comprising a foam sheet and showing an absorption capacity about 0.65 grams per cm² and a release of silver of 360 micrograms per cm² dressing when determined as disclosed below.

Another preferred embodiment of the invention is in the form of a dressing comprising an alginate material and showing an absorption capacity about 0.22 grams per cm² and a release of silver of 400 micrograms per cm² dressing.

A further preferred embodiment of the invention is in the form of a hydrogel showing a release of silver of 1000 micrograms per cm² dressing.

The skin-friendly adhesive may be any skin-friendly adhesive known per se, e.g. an adhesive comprising hydrocolloids or other moisture absorbing constituents for prolonging the time of use. The adhesive may suitably be of the type disclosed in those disclosed in U.S. Pat. Nos. 4,867,748 or 4,367,732.

The water impervious layer or film may be of any suitable material known per se for use in the preparation of wound dressings e.g. a foam, a non-woven layer or a polyurethane, polyethylene, polyester or polyamide film. A suitable film is e.g. the film disclosed in U.S. Pat. No. 5,643,187.

The dressing of the invention may have bevelled edges in order to reduce the risk of “rolling-up” the edge of the dressing reducing the wear-time and thus disturbing and prolonging the healing of the wounds. A bevelling may be carried out discontinuously or continuously in a manner known per se e.g. as disclosed in EP Pat. No. 0 264 299.

A protective cover or release liner may for instance be siliconized paper. It does not need to have the same contour as the dressing, e.g. a number of dressings may be attached to a larger sheet of protective cover. The protective cover is not present during the use of the dressing of the invention and is therefore not an essential part of the invention.

Furthermore, the dressing of the invention may comprise a “non touch” grip known per se for applying the dressing to the skin without touching the adhesive layer. Such a non-touch grip is not present after application of the dressing.

Suitable hydrocolloids for incorporation in the adhesive compositions of the invention are selected from naturally occurring hydrocolloids, semisynthetic hydrocolloids and synthetic hydrocolloids.

More particularly, the hydrocolloids are preferably selected from guar gum, locust bean gum (LBG), pectin, alginates, gelatin, xanthan and/or gum karaya; cellulose derivatives (e.g. salts of carboxymethylcellulose such as sodium carboxymethylcellulose, methylcellulose and hydroxypropylmethylcellulose) and/or sodium starch glycolate and/or polyvinylalcohol and/or polyethylene glycol.

In a second aspect, the invention relates to a method of enhancing healing of a wound comprising applying to the wound a dressing being capable of delivering an anti-microbially effective amount of silver ion activity to the wound bed and also being capable of removing wound exudate and matrix proteolytic enzymes from the wound bed.

The invention is now explained more in detail with reference to the below Examples describing preferred embodiments of the invention.

Materials and Methods

New-born Calf Serum (Lot. No.:118A) from Biochrom KG.

97% 5,5-Dimethyl-hydantoin (Commercially available from Aldrich) Water, distilled water from internal laboratory supply.

Purified Water (Demineralised water, conductivity 0.04 micros)

Ethanol 96% available from Danisco.

Hypol 2002 (An isocyanate prepolymer, commercially available from Dow Medical.

Pluronic 6200, a PO-PE block copolymer defoamer and surfactant from BASF

PEG 1000 Polyethylene glycol 1000, molecular weight 950-1050, available from Merck.

Aquapol 302-0019 a polyurethane prepolymer from Carpenter Co.

Silver nitrate powder (63.5% pure silver, commercially available from Johnson Matthey)

Sodium hydroxide (Analytical Grade, commercially available from Merck)

Sodium chloride (Analytical Grade, commercially available from Merck)

Sodium nitrate (Analytical Grade, commercially available from Merck)

Calcium chloride (Analytical Grade, commercially available from Merck).

Alphasan® RC 2000 Trade name for a Silver Sodium Hydrogen Zirconium Phosphate available from Milliken Chemical.

Actisorb Silver 220, a silver containing wound dressing from Johnson & Johnson Inc.

Acticoat, a silver containing wound dressing from Westaim Biomedical. AlgiSite M, a Calcium Alginate wound dressing from Smith+Newphew

Natrosol 250 HX, a hydroxyethyl cellulose (HEC) from Hercules

Determination of Absorption Capacity of a Sample

The absorption is measured in vitro by placing a sample of a size of 16 square centimeters in an excess of a solution of 1000 grams of distilled water from internal laboratory supply mixed with 142 mmol NaCl and 2.5 mmol CaCl₂ for 24 hours. After 24 hours, the sample is allowed to drip off for 1 minute and is re-weighed. The absorption capacity (g/cm²) is calculated from the difference in weight before and after absorption.

Determination of Release of Silver

The release of silver was determined by the following method.

Step A) The silver content of each sample was measured using a Spectro-XEPOS spectrophotometer from Spectro Analytical Instruments. Each determination was carried out in triplicate.

Step B) A sample of the material to be tested was cut in the shape of a disc having a diameter of 30 mm.

Step C) The sample was immersed in 50 ml of new born calf serum.

Step D) After stirring for 24 hours, the samples were removed from the liquid and, dried at 60° C. in a drying cupboard, and the remaining content of silver of the sample was measured using a Spectro-XEPOS spectrophotometer from Spectro Analytical Instruments. Each measurement was carried out in triplicate.

Step E) The loss of silver was calculated as weight of the Silver released from the dressing per square centimeters.

Preparation of Stabilized Silver Solution (SSS)

In 80 grams of purified water 18.5 grams of 5,5-dimethyl hydantoin, 4.1 grams of sodium hydroxide and 8 grams of silver nitrate was dissolved (the silver nitrate and the 5,5-dimethyl hydantoine were dissolved separately and mixed when the two solutions were clear to avoid precipitation). The solution was mixed with 920 grams of 96% Ethanol and 50 grams of PEG 1000. This solution was designated Stabilised Silver Solution (SSS). The concentration of silver in the SSS was app. 0.5% w/w.

### EXAMPLE 1

### Preparation of Antibacterial Foam Sheet

A polyurethane foam sheet was produced by mixing Hypol 2002 (10 grams), Aquapol (10 grams), Pluronic 6200 (0.2 grams), water (20 grams), Alphasan 2000 (3 grams) by first mixing the water, silver compound and Pluronic and then adding this mixture to the Hypol and Aquapol during mixing. While the mixture still was fluid it was transformed into thin layer by pouring the mixture onto a glass plate, placing a siliconised release paper on the mixture and adjusting the thickness to 2 mm using guiding bars and a doctor roll allowing the mixture to foam for several minutes. When the material was foamed, the foam sheet was dried in a dry air oven at 130° C. The final foamed sheet had a thickness of 4.5 mm and was cut into pieces of 10×10 cm, laminated to a polyurethane film, packed and sterilised using 30 kGy (beta irradiation). The foam sheet had a content of silver of 90 mg per dressing or 0.9 mg silver /cm² foam.

### EXAMPLE 2

### Preparation of an Antibacterial Alginate Fabric

An Alginate non woven fabric (Algisite M from Smith and Nephew) having the dimensions of 10×10 cm was immersed into SSS and allowed to absorb fluid until it was completely saturated (the fluid was absorbed within seconds). Then, surplus fluid was squeezed out of the alginate manually leaving 10 grams of absorbed fluid in the alginate. Finally the alginate was dried in an oven at 90° C. to a moisture content below 10% w/w (10 minutes). The Alginate had a silver content of 0.45 mg silver /cm² alginate or 45 mg per product. The final antibacterial alginate was packed and sterilised at 30 kGy using gamma irradiation.

### EXAMPLE 3

Preparation of an Antibacterial Amorphous Hydrogel

60 grams of Natrosol 250 HX was mixed with 920 grams of purified water and 20 grams of Alphasan 2000. The gel was put into 20 ml syringes and autoclaved. The silver concentration in the Hydrogel was 0.2% or 30 mg per sample (15 grams of gel in each sample).

### EXAMPLE 4

### Measurement of Absorption Capacity

The absorption capacity in vitro of various dressings of the invention prepared as disclosed in Examples 1-3 as compared to the commercially available Acticoat Dressing and Actisorb Silver Dressing was determined as disclosed above. The results are stated in the below Table 1.

TABLE 1

| | Foam | Alginate | Hydrogel | | Actisorb |
|---|---|---|---|---|---|
| Sample | (Ex. 1) | (Ex. 2) | (Ex. 3) | Acticoat | Silver |
| Absorption | 0.65 | 0.22 | NA* | 0.06 | 0.1 |
| (g/cm²) | | | | | |

*Not applicable (as the gel dissolves in the liquid and has no measurable area). Hydrogels are used on wounds which only secretes limited amounts or no exudate.

From the table above it can be seen, that the foam and alginate samples have higher absorption capacity than Acticoat and Actisorb Silver dressings.

### EXAMPLE 5

### Measurement of Release of Silver

The release of silver from various dressings of the invention prepared as disclosed in Examples 1-3 as compared to the commercially available Acticoat Dressing and Actisorb Silver Dressing was determined as disclosed above. The content of silver and the results are stated in the below Table 2.

TABLE 2

| | Foam | Alginate | Hydrogel | | Actisorb |
|---|---|---|---|---|---|
| Sample | (Ex. 1) | (Ex. 2) | (Ex. 3) | Acticoat | Silver |
| Ag Content | 900 | 450 | 1.000 | 1.200 | 20 |
| (myg/cm²) | | | | | |
| Ag-release | 390 | 400 | 1000* | 190 | 10 |
| (myg/cm²) | | | | | |

*½ gram gel per cm² (the gel dissolves in the serum)

From the table above it can be seen, that the foam, alginate and hydrogel samples of the invention have higher delivery of silver than Acticoat and Actisorb Silver dressings.

### EXAMPLE 6

### Result of Clinical Studies Using a Wound According to the Invention

In a test using 28 volunteers having chronic venous leg ulcers were treated with a foam dressing according to the invention for four weeks and the results were evaluated.

The purpose of the study was to investigate the performance profile of the dressing on wounds with bacterial problems identified by stopped or delayed wound healing, recurring wound infections or clinical signs such as heavy wound odor, increased sloughy exudation or plaque-like bacteria coverings.

75% of the wounds included suffered from stopped or delayed wound healing and almost 50% of them have had recurring wound infections. None of the wounds were clinically evaluated as infected at the inclusion of the study. The average size of the wounds was 14.2 cm² (1.3-41.5 cm²) and the average duration was 18 months (2-72 months).

The dressing was successful in initiating wound healing in these wounds being very difficult to heal. The overall reduction in relative wound area was 65% and the amount of granulation tissue in the wound increases from 32% to 83%. The odor from heavily smelling wounds was eliminated totally during the first week of treatment and exudation was decreased as well during the whole study period. The average wear-time of the dressing was 2.7 days. The absorption capacity of the dressing was evaluated as predominantly “good” and with very rare occasions of exudate leakage outside the dressing. The dressing was very easy to remove from the wound with no adherence to the wound tissue or any left over of residues. Peri-ulcer skin problems were reduced during treatment by the use of Conveen:Critic Barrier cream and the dressing in combination.

Thus, the following changes were observed indicating an onsetting healing of the chronic wounds:

Effective clearing of the wound bed, i.e. fast removal of slough and formation of granulation tissue

Promotion of healing compared to similar dressings without silver

Fast odour reduction

Reduced wound exudation

These findings were observed for patients with recalcitrant ulcers some with no healing progress for several years and clearly indicates the wound promoting effect of the dressings of the invention when treating chronic ulcers.

1. A medical dressing comprising a silver compound and being capable of releasing antimicrobial silver ion activity to a wound and, at the same time, being capable of absorbing wound exudate and also degrading enzymes from the wound.

2. A medical dressing as claimed in claim 1 wherein the dressing comprises the silver compound in the form of a complex stabilizing the silver against reduction to free silver.

3. A medical dressing as claimed in claim 2 wherein the dressing comprises the silver in the form of a complex comprising silver and an element of Group IVa of the periodic system.

4. A medical dressing as claimed in claim 2 wherein the dressing comprises the silver in the form of a complex with a primary, secondary or tertiary amine which complex is associated to one or more hydrophilic polymers.

5. A medical dressing as claimed in any of claims 1-4 wherein the dressing comprises absorbing constituents in the form of an individual part of the dressing or in the form of a discontinuous phase distributed in an adhesive matrix.

6. A dressing as claimed in claim 5 wherein the absorbing constituent is in the form of hydrocolloid particles distributed in an adhesive matrix.

7. A dressing as claimed in claim 5 wherein the absorbing constituent is in the form of an element of an absorbing foam material.

8. A dressing as claimed in claim 5 wherein the absorbing constituent is in the form of an element of an alginate material.

9. A method of enhancing healing of a wound comprising applying to the wound a dressing being capable of delivering an anti-microbially effective amount of silver ion activity to the wound bed and also being capable of removing wound exudate and degrading enzymes from the wound bed.
